(19) United States
(12) Patent Application Publication
(10) Pub. No.: US 2024/0078291 A1
(43) Pub. Date: Mar. 7, 2024

(54) SYSTEMS AND METHODS FOR MANAGING, PROVIDING, OR APPLYING MILITARY, FORENSICS, OR RELATED INTELLIGENCE

(71) Applicants: Jonathan Grier, Owings Mills, MD (US); Justin Phillips, Owings Mills, MD (US); Dane Howard, Owings Mills, MD (US); Ben Marshall, Owings Mill, MD (US)

(72) Inventors: Jonathan Grier, Owings Mills, MD (US); Justin Phillips, Owings Mills, MD (US); Dane Howard, Owings Mills, MD (US); Ben Marshall, Owings Mill, MD (US)

(21) Appl. No.: 18/243,661

(22) Filed: Sep. 8, 2023

Related U.S. Application Data
(63) Continuation of application No. 18/243,660, filed on Sep. 7, 2023.
(60) Provisional application No. 63/374,776, filed on Sep. 7, 2022.

(51) Int. Cl. G06F 18/23213; G06F 18/2413

(52) U.S. Cl. CPC G06F 18/23213; G06F 18/24147

(57) ABSTRACT

Apparatus, systems and methods are provided that create an improved forensic investigation graph. Nodes of connected data are clustered according to a maximal nearest neighbor algorithm to create maximal nearest neighbor clusters. A first node of data is directly connected to at least a second node of data and indirectly connected to a third node of data through the second node. The nearest neighbor includes only sets of nodes that are directly connected. A cluster of data includes combinations of connected nodes. A cluster of nearest neighbors only includes combinations of nodes that are directly connected to each other. The maximal nearest neighbor clusters are created by determining all clusters or nearest neighbors and removing all nearest neighbor clusters that are subsets of another nearest neighbor cluster. The maximal nearest neighbor clusters re then displayed on a display. The maximal nearest neighbor clusters represent data acquired in the performance of a forensic investigation.

### CROSS REFERENCE TO RELATED APPLICATIONS

This application claims the benefit of the filing date of U.S. Provisional Patent Application Ser. No. 63/374,776, entitled: “Improved Systems and Methods for Managing, Providing, or Applying Military, Forensics, or Related Intelligence,” which was filed in the USPTO on Sep. 7, 2022 and which includes the same inventors. That provisional application is hereby incorporated by reference as if fully set forth herein.

### STATEMENT OF FEDERALLY SPONSORED RESEARCH OR DEVELOPMENT

This invention was made with government support under contract No. M6785420C6704 awarded by Commander Marine Corps System Command. The government has certain rights in the invention.

### FIELD OF THE TECHNOLOGY

The technology of the application relates generally to improved forensic investigations and more specifically, but not exclusively to apparatus, systems and methods which leverage machine learning to locate common patterns in unrelated and/or related case files and present those common patterns in a forensic investigation to improve the efficiency and quality of the investigation.

### BACKGROUND OF THE TECHNOLOGY

A forensic investigation is the gathering and analysis of evidence to assist in proving or disproving a particular action was cause by a particular suspect and/or to assist in identifying a suspect. A suspect may be human, animal, virus or some other actor that is the cause and/or assisted in the cause of the action. Evidence may include blood, other fluids, fingerprints, residue, computers, hard-drives, phones, other technologies, irregularities in accounting or other data, images, biometric data, etc. In other words, evidence may be any clue that assists with the identification or ruling out of a suspect or of other evidence.

Since different forensic investigations may be performed by different people, in different jurisdictions, at different times and/or for different reasons, forensic investigations may be related to one another without the investigators being aware that other related investigations are taking place or have taken place. Forensic investigations may not be entirely related, yet they may share common evidence. Knowledge of that common evidence may assist in providing solutions in one or more of the investigations. Additionally, in certain investigations, such as military or terrorism related investigations, time may be of the essence and the faster investigators can resolve the investigation the more likely the authorities may be to capture a suspect/perpetrator.

In view of these deficiencies in conventional forensic investigations, the instant disclosure identifies and addresses a need for systems, apparatus and methods which improve forensic investigations by providing evidence to investigators that may not have otherwise been brought to their attention and/or in a more efficient manner.

### BRIEF SUMMARY OF THE TECHNOLOGY

Many advantages of the technology will be determined and are attained by the technology, which in a broad sense provides systems apparatus and methods for improving forensic investigations by identifying common evidence from related and/or unrelated cases to an investigator.

In one or more implementations of the technology, a computer-implemented method is provided for creating an improved forensic investigation graph, at least a portion of the method being performed by a computing device that has at least one processor. The method includes clustering nodes of connected data according to a maximal nearest neighbor algorithm to create maximal nearest neighbor clusters. A first node of data is directly connected to at least a second node of data and indirectly connected to a third node of data through the second node. The nearest neighbor includes only sets of nodes that are directly connected. A cluster of data includes combinations of connected nodes. A cluster of nearest neighbors only includes combinations of nodes that are directly connected to each other. The maximal nearest neighbor clusters are created by determining all clusters or nearest neighbors and removing all nearest neighbor clusters that are subsets of another nearest neighbor cluster. The method further includes displaying the maximal nearest neighbor clusters on a display associated with the computing device. The maximal nearest neighbor clusters represent data acquired in the performance of a forensic investigation.

In one or more implementations of the technology, a non-transitory computer-readable medium is provided that may include one or more computer-executable instructions that, when executed by at least one processor of a computing device, cause the computing device to cluster nodes of connected data according to a maximal nearest neighbor algorithm to create maximal nearest neighbor clusters. A first node of data is directly connected to at least a second node of data and indirectly connected to a third node of data through the second node. A nearest neighbor includes only sets of nodes that are directly connected. A cluster of data includes combinations of connected nodes. A cluster of nearest neighbors only includes combinations of nodes that are directly connected to each other, and the maximal nearest neighbor clusters are created by determining all clusters or nearest neighbors and removing all nearest neighbor clusters that are subsets of another nearest neighbor cluster. The instructions also cause the computing device to display the maximal nearest neighbor clusters on a display associated with the computing device.

Features from any of the above-mentioned embodiments and/or examples may be used in combination with one another in accordance with the general principles described herein. These and other embodiments, features, and advantages will be more fully understood upon reading the following detailed description in conjunction with the accompanying drawings and claims.

### BRIEF DESCRIPTION OF THE DRAWINGS

For a better understanding of the technology, reference is made to the following description, taken in conjunction with the accompanying drawings, in which like reference characters refer to like parts throughout, and in which:

FIG. 1 illustrates an exemplary architecture of the disclosed technology;

FIG. 2 illustrates an exemplary acquisition stage of FIG. 1 in accordance with one or more embodiments of the technology;

FIG. 3 illustrates an exemplary examination stage of FIG. 1 in accordance with one or more embodiments of the technology;

FIG. 4 illustrates an exemplary matching stage of FIG. 1 in accordance with one or more embodiments of the technology;

FIG. 5 illustrates an exemplary associating stage of FIG. 1 in accordance with one or more embodiments of the technology;

FIG. 6 is an exemplary diagram of node connections;

FIG. 7 illustrates exemplary pseudo-code for performing maximal nearest neighbor clustering in accordance with one or more embodiments of the technology;

FIG. 8 is a more complex exemplary diagram of node connections;

FIG. 9 provides a block diagram illustrating all nearest neighbor clusters for the node connections in FIG. 8, in accordance with one or more embodiments of the technology;

FIG. 10 provides a block diagram illustrating all nearest neighbor clusters for the node connections in FIG. 8 that are subsets of another cluster, in accordance with one or more embodiments of the technology;

FIG. 11 provides a block diagram illustrating all remaining nearest neighbor clusters for the node connections in FIG. 8 after removing all clusters that are subsets of another cluster, in accordance with one or more embodiments of the technology;

FIG. 12 provides a block diagram illustrating prior art technique of maximal cliques for the node connections in FIG. 8 after applying Bron-Kerbosh;

FIG. 13 provides an exemplary dashboard display showing active cases on which an analyst might be working in accordance with one or more embodiments of the technology;

FIG. 14 provides an exemplary link analysis corresponding to an active case from FIG. 13 in accordance with one or more embodiments of the technology;

FIG. 15 provides an exemplary selection of an information reference from FIG. 14 displaying additional information in accordance with one or more embodiments of the technology;

FIG. 16 provides an exemplary indication of a number of remaining references to be displayed for a particular list of common items of evidence between case files according to FIG. 15, in accordance with one or more embodiments of the technology;

FIG. 17 illustrates the ability to access the additional hidden references from FIG. 16 in accordance with one or more embodiments of the technology;

FIG. 18 illustrates further navigation down the chain of potentially related case files of FIG. 14 in accordance with one or more embodiments of the technology;

FIG. 19 illustrates further navigation down the chain of potentially related case files of FIG. 18 in accordance with one or more embodiments of the technology;

FIG. 20 provides an exemplary grid view of the dashboard of FIG. 13 in accordance with one or more embodiments of the technology;

FIG. 21 is a zoomed-in view of the tabs from FIG. 20 illustrating the ability to toggle between views in accordance with one or more embodiments of the technology;

FIG. 22 provides an exemplary grid view of a selection of an information reference from FIG. 20 displaying additional information in accordance with one or more embodiments of the technology;

FIG. 23 provides an exemplary grid view of information contained in the primary case file selected from FIG. 20, in accordance with one or more embodiments of the technology; and,

FIG. 24 provides an exemplary selection of an information reference from FIG. 22 displaying additional information in a separate window in accordance with one or more embodiments of the technology.

The technology will next be described in connection with certain illustrated embodiments and practices. However, it will be clear to those skilled in the art that various modifications, additions, and subtractions can be made without departing from the spirit or scope of the claims.

### DETAILED DESCRIPTION OF THE INVENTION

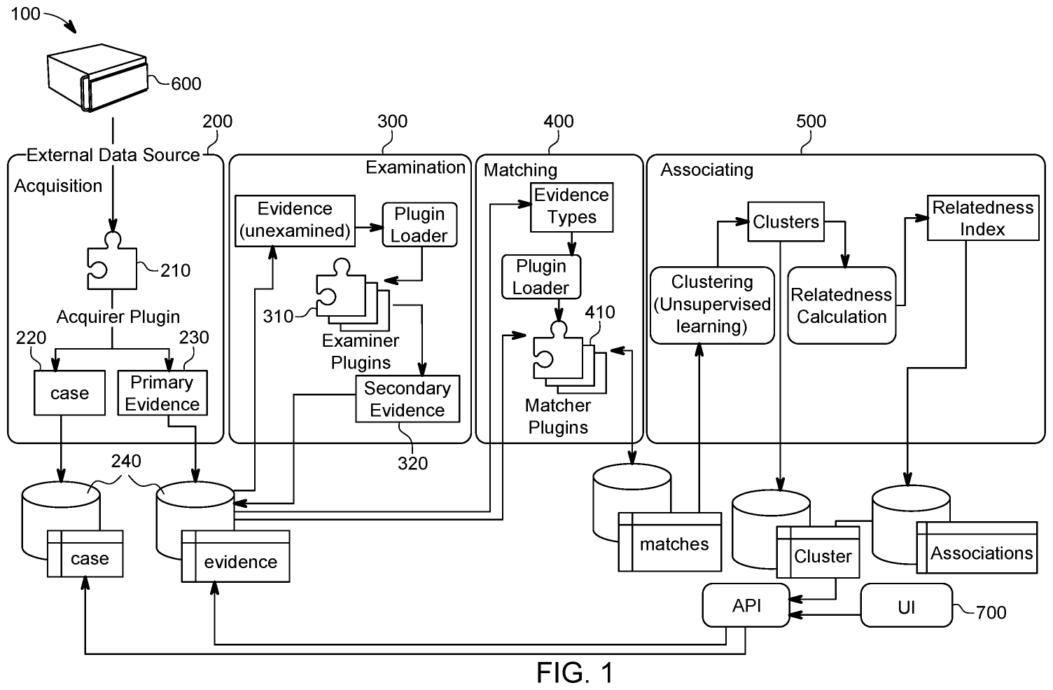

Referring to the drawings in detail wherein like reference numerals identify like elements throughout the various figures, there is illustrated in FIGS. 1-11 and 12-24 apparatus, systems and methods for improving forensic investigations by leveraging machine learning to locate common patterns in unrelated and/or related case files and presenting those common patterns to the investigator. Principles and operations of the technology may be better understood with reference to the drawings and the accompanying description.

Discussion of an embodiment, one or more embodiments, an aspect, one or more aspects, a feature, one or more features, or a configuration or one or more configurations, an instance or one or more instances is intended be inclusive of both the singular and the plural depending upon which provides the broadest scope without running afoul of the existing art and any such statement is in no way intended to be limiting in nature. Technology described in relation to one or more of these terms is not necessarily limited to use in that embodiment, aspect, feature or configuration and may be employed with other embodiments, aspects, features and/or configurations where appropriate.

The technology provides a system that leverages plugin modules supplemented by machine learning for a robust and adaptable system that obtains data from one or more data sources, separates the data into smaller units of data (e.g., deconstructs the data into its smallest logical data items/fields) determines common data types within the smaller units, creates “edges” that match data from the one or more data sources with data already in the system, then determines potentially overlapping clusters of information to identify potential connections between investigations. A cluster is when different cases are sufficiently related to each other. In other words matching across cases or between cases may result in common patterns or data. The technology may draw connections between cases, which may be visually displayed in a link view or otherwise presented to the analyst in a useful manner.

Each plugin module performs a limited set of functions thus providing the ability of the system to be quickly modified using proprietary software and/or off-the-shelf software wrapped to integrate into the system. A plugin module may be an open source or proprietary module (e.g., a module to perform machine learning, natural language processing, facial recognition, etc.) and may be wrapped by software and plugged into and used by the framework. The sufficiency of a plugin may depend on if it conforms to a set of predefined rules. The modularity of the system allows the plugin registry to be modified to adapt to specific applications without changing the source code. The system may be returned to the core functions quickly and easily after being modified for an application, providing system stability. Further, the plugins can function in parallel and/or sequentially thus providing efficiency and speed and they enable the system to be upgraded without having to redesign the entire system. While the plugins have been described as modular, one or more plugins may be permanently integrated into the system and still fall within a scope of one or more of the claims of this application.

The technology may perform some or all the initial “grunt work” automatically, so the analyst may move on to deeper analysis work more quickly and more confidently. In one or more embodiments, the technology performs a partial analysis of the data and allows the analyst to determine if additional analysis is required/warranted. In such embodiments, a human user may parse through remaining or underlying information and make relevant decisions, as discussed further below. In this manner the technology may support and help guide the human investigator in the field. In one or more embodiments, the technology may leverage unsupervised machine learning to perform a link analysis. The leveraging of unsupervised machine learning contrasts with supervised machine learning. Nevertheless, the technology can also be extensible, and in other examples the technology may leverage pre-trained, supervised machine learning, natural language processing, facial recognition, etc.

FIG. 1, illustrates an exemplary architecture 100 of the technology. As illustrated in FIG. 1, the technology may provide a modular forensic investigation tool that may be realized as a hardware computing device (not shown) loaded with various software modules that perform the various functions discussed herein. An external data source may be connected to the hardware via conventional connectors to conventional ports on the hardware device. In one or more embodiments, the hardware device may be connected to the data source via a network. While not preferred or recommended, the network may be a public wide area network such as the Internet and still fall within a scope of one or more of the claims. Preferably, if the hardware is connected to the data source via a network, it is via a private local area network (LAN) or private wide area network (WAN). Additionally, or alternatively, the system may be realized at least in part as software that is loaded onto the data source. As illustrated in FIG. 1, the system operates in multiple stages; namely Acquisition 200, Examination 300, Matching 400, and Association 500.

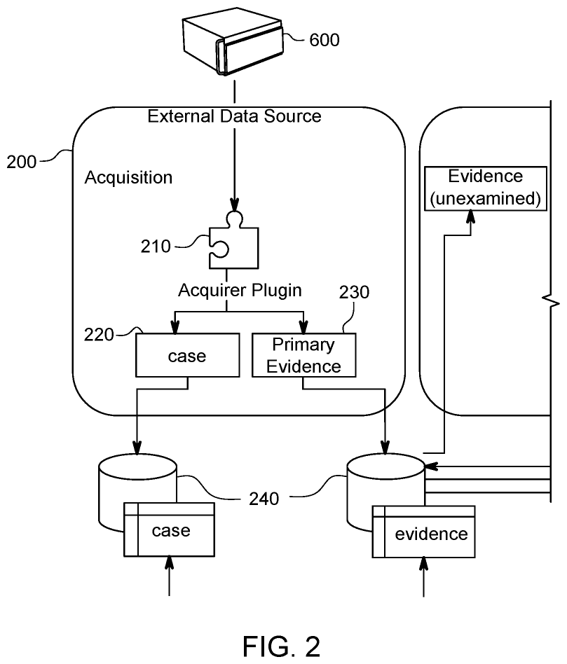

FIG. 2 illustrates an exemplary Acquisition stage 200 in which one or more modules (also referred to as acquirer plugins) 210 working sequentially and or in parallel copies/copy data from the data source. Since the system may be designed to work with various data sources, different modules 210 may be employed. While the figures only illustrate a single module 210, that is not intended to be a limiting factor. Multiple modules 210 may be employed. For example, one or more modules 210 may be dedicated to a particular hardware type, such as but not limited to mobile phones, computers, cameras, storage devices, etc. Additionally, or alternatively, one or more modules 210 may be dedicated to a particular file type such as text, image, video, audio, etc.

Module 210 may be realized as software written specifically for the system 100 and/or it may include commercial software wrapped to enable it to seamlessly interact with the system 100. Techniques for wrapping software to enable plug-and-play operation are conventional and thus will not be further described. Module 210 consumes data from a data source 600 and enters it into system 100. Module 210 may consume data via monitoring the data source 600 (in real-time or otherwise), it may perform one or more searches of the data source 600 for specific information and/or data formats, it may monitor and/or search only specific defined portions of the data source and/or it may monitor one or more portions of the data source while searching other portions of the data source and/or it may monitor and/or search different portions of the data source at random and/or scheduled times. Module 210 receives data and catalogs the data according to predefined data structures. In one or more embodiments, module 210 stores the various data structures within one or more databases 240 or some other acceptable storage file 240.

In one or more embodiments, a module 210 may operate upon digital files. As such, the technology may operate at the level of folder structures, such that each folder may correspond to a case file. In one or more embodiments, a module 210 may operate on any suitable database. Additionally, or alternatively, when a module 210 is extracting data from a computer, the computer could be connected to the system and all corresponding cases may be extracted from that computer. Thus, for any suitable data source, a corresponding plugin module 210 may be ascertained, acquired, or created and then employed for extracting data from that data source 600. By separating the functions into separate parts, the user or analyst may be provided the ability to input data from various heterogeneous technologies or formats and convert this data into a consistent format and perform analysis on the backend.

An illustrative example of the Acquisition Phase 200 may include a military law enforcement officer (MLEO) who possesses a laptop that is used to manage the MLEO's case data. The MLEO may store files into separate folders of a file system on that laptop and designate each folder as a single case. In one or more embodiments, module 210 monitors (e.g., in real-time) one or more of the folders and extracts copies of the data as it is being input or stored. In one or more embodiments it then creates case files 220 and primary evidence files 230. In one or more embodiments, only one form of data structure may be created while in one or more embodiments, multiple forms of data structures may be created by module 210. Additionally, each separate data structure may be stored within a corresponding database 240 or some other acceptable storage file 240.

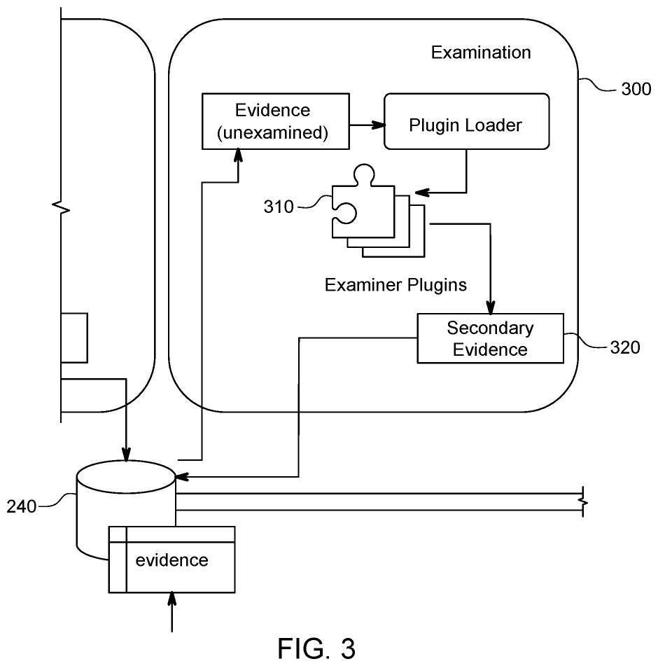

FIG. 3 illustrates an exemplary examination stage 300. After the acquisition stage acquires data from one or more data source(s) 600, the system may include data extracted from heterogeneous data sources 600. For example, there may be biometric enrollment files that are stored within a case file and there may be a report extracted from a phone (e.g., using a tool such as Cellebrite™), and there may be other data. While in a preferred embodiment all the primary evidence has been stored in the acquisition stage 200 prior to the examination stage 300 performing its operations, in one or more embodiments the data may be examined on an ongoing basis or in stages. Further, while the system may only include data extracted from homogenous data sources 600, the remaining description will be limited to data extracted from heterogeneous data sources 600 as that description will be sufficient for understanding the homogeneous situation as well.

At the examination stage 300, one or more modules (also referred to as examiner plugins or plugins) 310 determines the type of file corresponding to each instance of data and how to parse that data. Further, in one or more embodiments, an examiner plugin 310 may parse data stored in the acquiring stage 200 into smaller units of data (one time or recursively) and store those smaller units of data as secondary evidence 320. In one or more embodiments, each module 310 may be configured to parse a specific set of evidence type and/or corresponding data structure (e.g., phone number, fingerprint, biometric enrollment file, textual string, etc.). Each examiner plugin 310 may be modular such that it does not need to be natively integrated into the framework, but instead can be plugged in or out. Similarly, each module 310 does not need to be created or derived from the same source, or even from the same source as the forensic tool. Instead, the tool may use modules 310 created by different sources, such as open-source plugins 310, proprietary plugins 310, third-party plugins 310, and/or native and inhouse plugins 310, etc. Regardless of the source or origin of each plugin 310, the plugin 310 may be modularly inserted or removed, and potentially replaced, etc.

By parsing data into smaller and smaller units, the examination phase 300 identifies instances of data of the same type (e.g., text strings, phone numbers, facial recognition results, etc.). In other words, beginning with different types of data (e.g., data extracted from a cell phone, a biometric enrollment file, etc.), the examination stage 300 may extract small units of data from the data stored by acquisition stage 200 until the small units of data are separated into specific types. For example, the data extracted from a cell phone might reveal, through acquisition 200 and examination 300, a picture featuring a face that is identified through facial recognition. Additionally, a biometric enrollment file may also reveal the same face for an individual enrolled using the biometric enrollment file. Each of these images will be parsed from the original data into an image file.

The iterative extraction, of parsed items of nested information, may be performed recursively. As an illustrative example, evidence A produces evidence B which produces evidence C. This may be performed by calling the same or essentially the same method on evidence B that was previously called on evidence A. Thus, at different layers of the nested evidence extraction process, the technology may identify that an item of extracted information has a different type than another item of extracted information. Accordingly, the technology may load one or more plugins 310 corresponding to the different types of extracted information respectively, thereby breaking down the information into its different parts and processing the different parts accordingly.

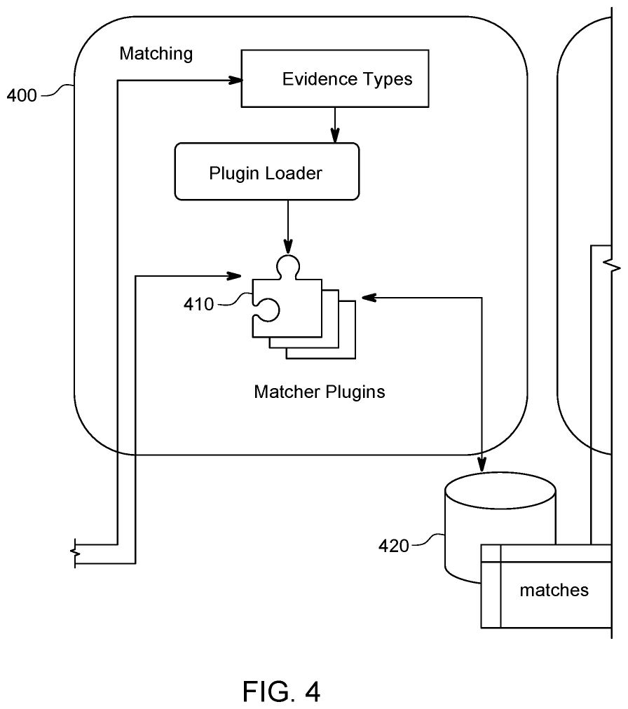

FIG. 4 illustrates an exemplary matching stage 400. During matching stage 400 one or more modules (also referred to as matcher plugins) 410 determine matches amongst the parsed like units of data extracted from examination stage 300 (e.g., match names to names, faces to faces, phone numbers to phone numbers, etc.). In one or more embodiments one or more options/configurations may exist for matching larger portions of information (i.e., mixed data types). In such configuration(s) the system may employ vectors, fuzzy logic, or any other conventional form of matching to determine the level of similarity between the information. Such a configuration may sacrifice completeness for speed, which in certain instances may be more important. The match information may be stored in a database 420 or some other acceptable storage file 420.

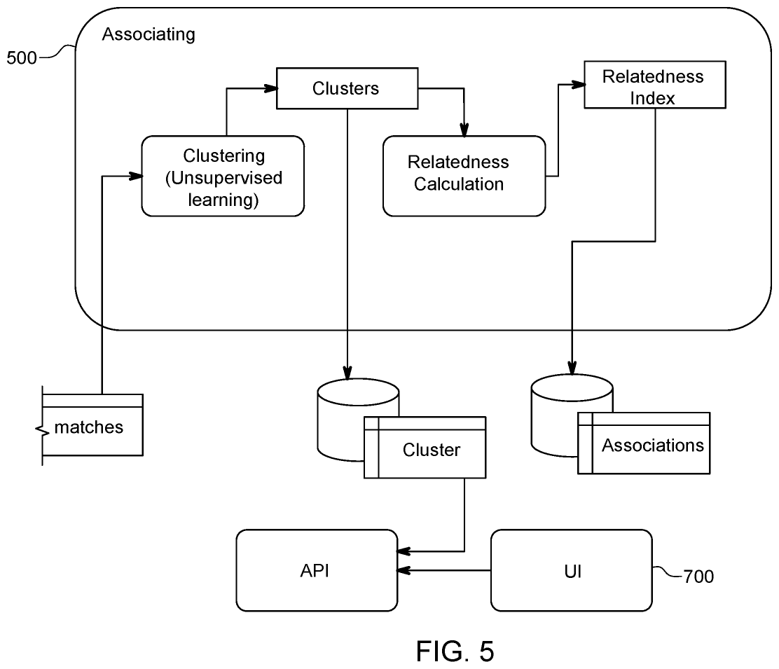

FIG. 5 illustrates an exemplary associating or clustering stage 500. In stage machine learning may be applied to create clusters. The following discussion provides an overview of a clustering algorithm, which may be performed at clustering stage 500. For ease of description, the algorithm may be titled a “maximal nearest neighbor” clustering algorithm, which may correspond to a novel graph algorithm to prevent overfitting. In one or more examples, the clustering algorithm may correspond to a within-graph clustering algorithm that subdivides a single graph into clusters within that particular graph (as distinct from a between-graph clustering algorithm).

In one or more examples, the clustering algorithm may correspond to a deterministic graph clustering algorithm that avoids “chaining” and is tolerant of overlapping clusters. The clustering algorithm may be useful for clustering graphs that are densely connected between related vertices and loosely or unconnected between more unrelated vertices. The clustering algorithm may thereby avoid the “chaining” phenomenon, which is a phenomenon whereby new nodes are added to a cluster because they are close to at least some of the nodes in the current cluster despite possibly being quite far from others, as discussed in more detail below.

The following describes certain conventional clustering algorithms, which the maximal nearest neighbor clustering algorithm may improve upon. The K-spanning tree algorithm may require a desired number of clusters (i.e., K) to be known beforehand. Nevertheless, analysts do not necessarily know the number of clusters to be included before running the algorithm. The number of clusters needs to be driven by the quality of the matches.

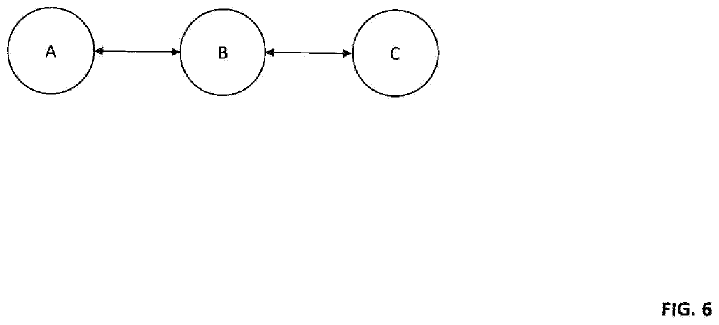

The shared nearest neighbor clustering algorithm may denote edge weight based on a number of edges common between two nodes. This algorithm does not necessarily provide enough flexibility in terms of not allowing clusters to overlap. In the case of FIG. 6, when utilizing this algorithm, the only possible clusters are (A,B,C) or (A,B), (C), or (A,C) (B), or (B,C) (A), or (A), (B), (C). Accordingly, the algorithm does not provide the electability to overlap clusters (i.e., (A,B), (B,C)). As further shown in FIG. 6, in this example A is close to B, which is close to C, and yet C is not close to A, and for this reason all three nodes may be pulled into the same big cluster, which creates the problem of chaining (i.e., A, B, and C are chained together, even though A and C would more preferentially not be connected to each other). In contrast, the more desirable solution corresponding to the improved algorithm described herein may prevent A from being associated with C. At the same time, it would not be desirable to just cluster A and B together, and then leave C separate, because this would ignore the close relationship between B and C. Accordingly, a more desirable or improved solution would allow overlapping clusters, such as (A,B) and (B,C).

Another clustering algorithm may correspond to highly connected subgraph clustering. In this example, a graph may be determined to be highly connected if the maximum number of edges required to separate the graph into two subgraphs is greater than the number of vertices divided by two. If the graph is highly connected, it is not separated any further. The process is then repeated recursively on each subgraph until only highly connected clusters remain. This algorithm may suffer from the same flexibility deficiency as the shared nearest neighbor clustering algorithm discussed above.

As another example, a Louvain method for community detection, in the context of the use case described herein, may have the tendency to perform “overfitting” that could not be controlled. “Overfitting” can refer to a clustering algorithm's tendency to grow the largest cluster aggressively, thereby causing the largest cluster to contain all connected vertices, even though this can destroy all of the potential resolution in the clusters.

As an additional example, K-nearest neighbors clustering may require a set number (i.e., K) of clusters. However, an analyst may not necessarily know the number of clusters before executing the algorithm. Accordingly, although one could dynamically select a value for K, this algorithm will tend to result in the overfitting problem that is further discussed above due to the “over chaining” problem.

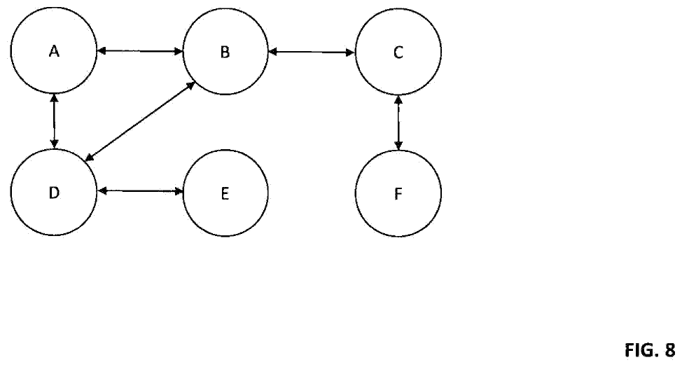

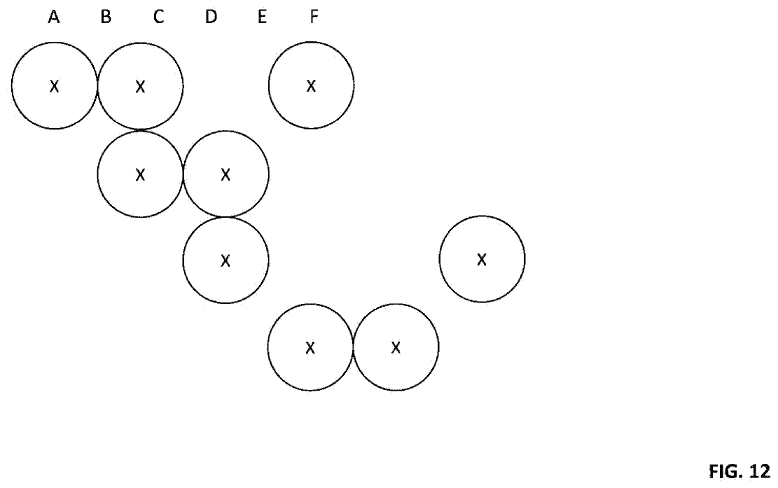

Additionally, a maximal clique enumeration algorithm may create clusters that are “maximal cliques” found using the “Bron and Kerbosh Algorithm” for finding such clusters. This is illustrated by FIGS. 8 and 12. A “clique” is a maximal complete subgraph, or a complete subgraph that is not contained in any other complete subgraph. In contrast, the maximal nearest neighbor clustering algorithm of this application does not have the requirement that all “clusters” must be complete.

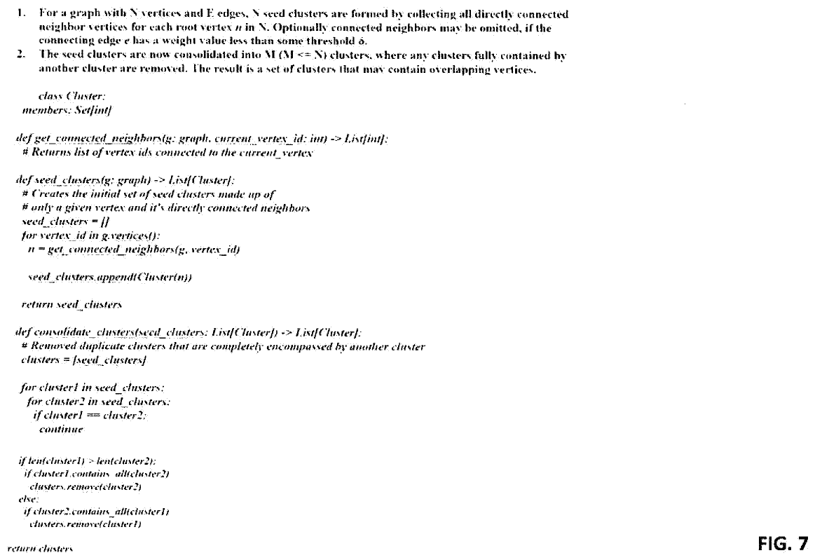

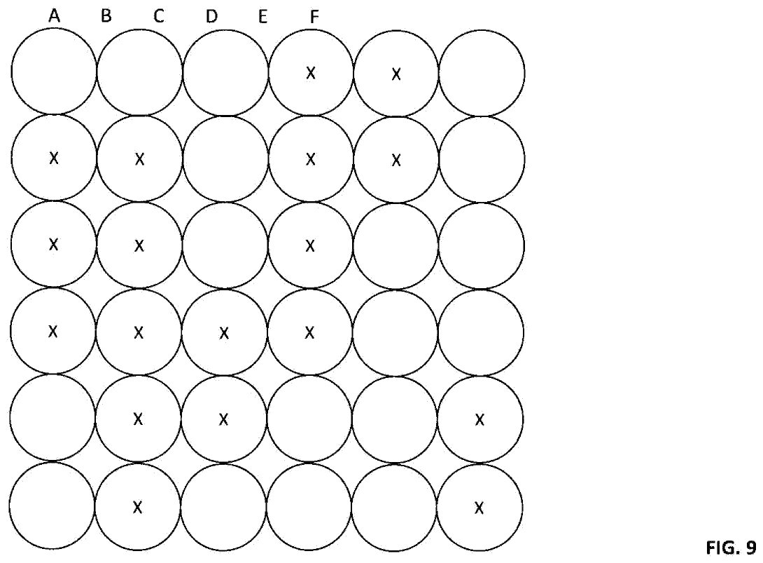

Returning to the maximal nearest neighbor clustering algorithm, FIG. 7 provides illustrative pseudocode to perform this clustering algorithm. The maximal nearest neighbor algorithm may begin by creating a single seed cluster for each node on the graph. So, in the example provided in FIGS. 8-11, six separate graphs would initially be created. Thus, with six nodes (FIG. 8), one would create six clusters (FIG. 9). By way of example, the first cluster for node A also features B and D, because B and D are nearest neighbors of A. Similarly, the cluster for node C would feature B and F because these are the nodes that are the nearest neighbors of node C, and so on, as illustrated in FIG. 9.

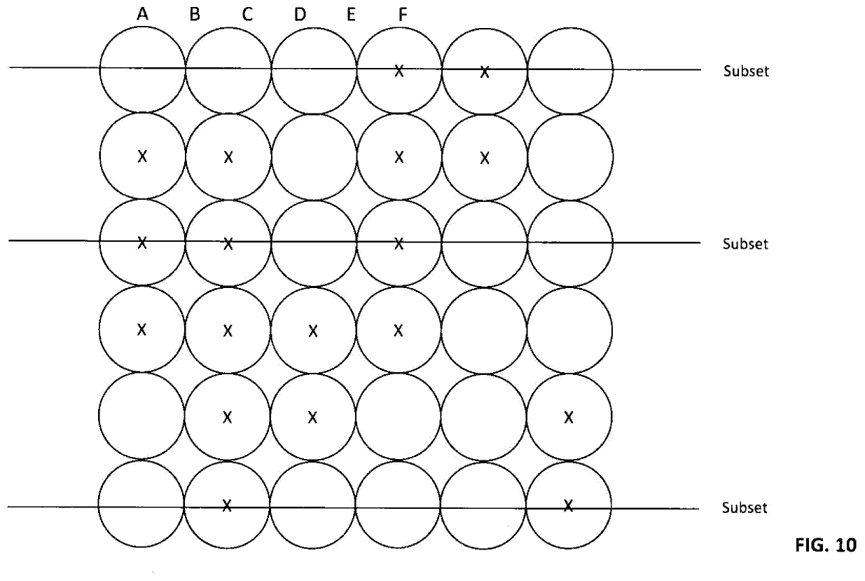

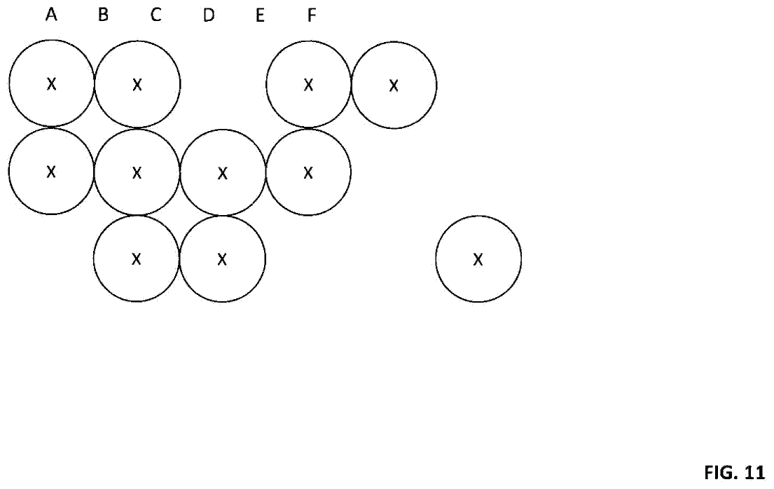

After this initial step, the maximal nearest neighbor clustering algorithm may proceed by removing all clusters that are subsets of another cluster (FIGS. 10 and 11). Thus, as illustrated in FIG. 10, the cluster (A,B,D) (corresponding to original node A) is entirely contained within (B,A,C,D) (corresponding to the original node B), and for this reason (A,B,D) can be removed as redundant. Similarly, the cluster (F,C) (corresponding to original node F) is entirely contained within the cluster (C,B,F) (corresponding to original node C), and therefore cluster (F,C) can be removed. It should be noted that, in the results of the maximal nearest neighbor clustering algorithm in FIG. 11, there are spaces between separate nodes, however, the spaces are simply used to make parallel nodes overlapping in the vertical dimension and do not indicate disconnections between clusters, rather the clusters are identified by row (i.e., the three clusters (A,B,C,D), (B,C,F), and (A,B,D,E)).

To further clarify, in the example illustrated in FIGS. 8-11, each one of the nodes being processed by the maximal nearest neighbor clustering algorithm may correspond to parallel types of data. So, in such examples, each of the nodes being processed might all correspond to fingerprints, or might all correspond to names, or might all correspond to phone numbers, or might all correspond to mission case files, or might all correspond to biometric enrollment files, and so on. In other words, each one of the nodes may correspond to an atomic node or lowest level item of evidence resulting from the recursive, de-nesting, or otherwise extracting of items of nested information or evidence, as further discussed above. As also discussed above, it is possible that the above nodes may include higher levels of information such as but not limited to name and address or name and image and they may not be exact matches (e.g., a node may include name that matches name but an address that does not match).

Returning to FIG. 5, after the creation of the clusters, a relatedness calculation may be performed, which may calculate the degree to which different cases share entities in the same cluster, based on term frequency inverse document frequency (tf-idf). In other examples, in the context of information retrieval, any other suitable numerical statistic or substitute statistical analysis may be performed to rank entities in cases in terms of how important or relevant they are to that particular case, or to otherwise identify relevant relationships (e.g., one or more of word embedding, Kullback-Leibler divergence, latent dirichlet allocation, latent semantic analysis, mutual information, noun phrase, Okapi BM25, PageRank, vector space model, word count, and/or SMART information retrieval system, etc., as appropriate). Such evaluation may be performed with respect to each cluster. Moreover, using those rankings, the associating stage 500 may determine which cases are most associated with which other cases, thereby generating a link view for presenting to an investigator.

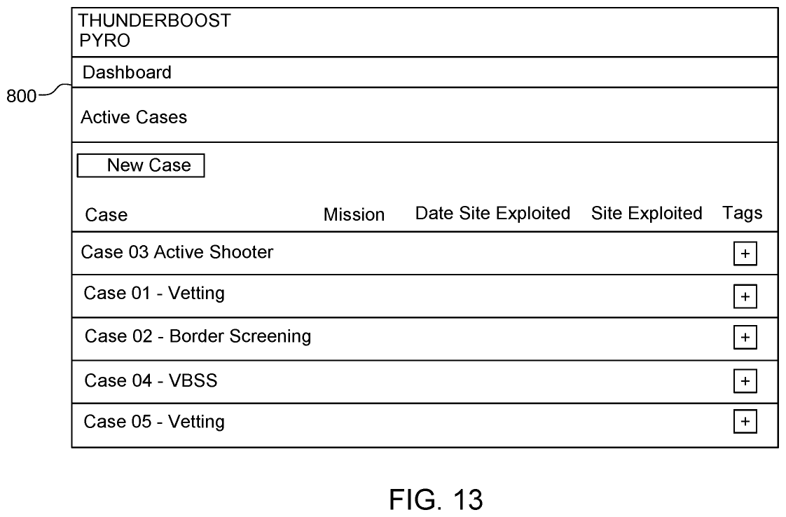

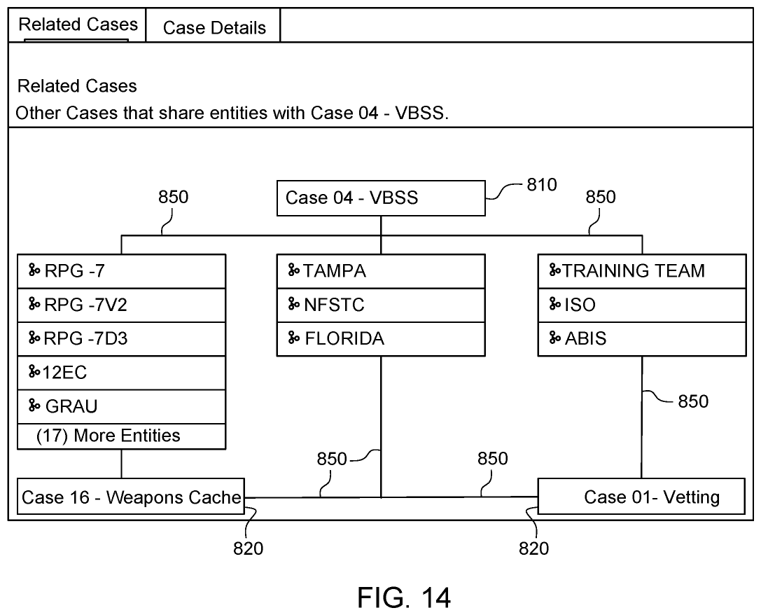

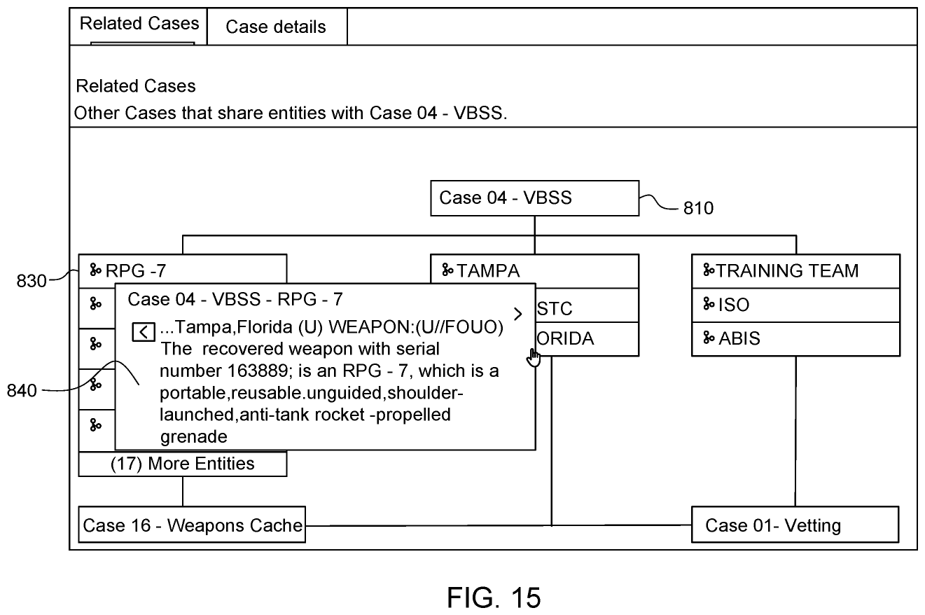

Once the information is acquired, broken down and matched, it can be presented on a user interface (UI) 700 that allows the analyst to perform a detailed review. FIGS. 13-24 illustrate an exemplary UI in accordance with one or more embodiments of the technology. FIG. 13 illustrates dashboard 800 showing active cases 1-5 on which an analyst might be working. FIG. 14 illustrates an exemplary link analysis corresponding to Case 04—VBSS in FIG. 13. The analyst may only be considering one case (e.g., Case 04), but there may be other cases that relate to this case. In this example, case 04 is displayed at a particular position (e.g., the top) that indicates that it is the primary or current case 810 being viewed, whereas the remaining cases 820 are shown as being related to this case. In other words, in response to the user input selecting a specific node, a different view of the forensics case graph is displayed emphasizing that specific node. FIG. 15 illustrates that in this example, the technology parsed text documents and obtained a reference 830 to an RPG-7, which is a type of weapon. When the analyst selects this reference 830 additional information 840 may be displayed. While the nodes are illustrated in a particular configuration, those skilled in the art will recognize that this is merely a design choice and any configuration that imparts the same information in a meaningful manner may be employed without departing from a scope of the technology. For example, rather than being top down the display could be configured right to left or left to right or hub and spoke, etc.

As illustrated in FIG. 15, this reference 830 was found in both case 04 and case 16. Thus, an operator may ascertain from the display, that the reference 830 suggests that these cases are related or similar. Alternatively, the analyst may decide that more information is needed to make that determination and thus the analyst may select the additional common references 830 to make the determination. As best seen from FIG. 14, rather than simply arranging the information on the display, the display may also provide links 850 between the case files showing which case files share common patterns and/or data. This provides the analyst with a lot of information in an easily digestible format that can be accessed and analyzed quickly and efficiently. This is particularly important when time is of the essence in an investigation.

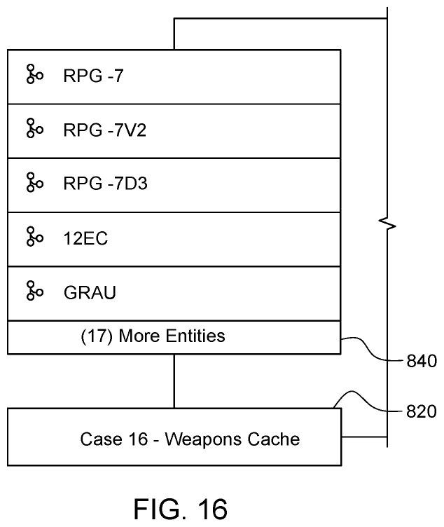

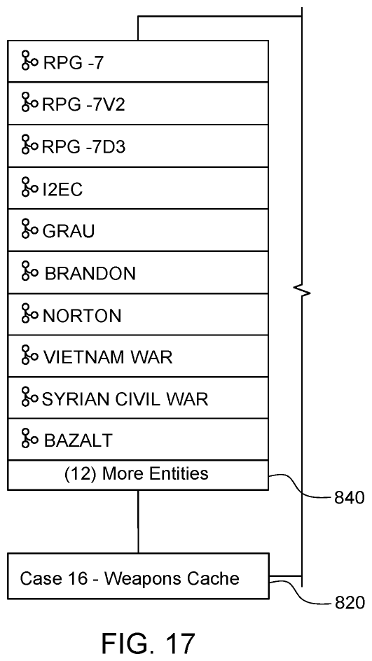

As illustrated by FIGS. 16 and 17, in one or more embodiments the display may provide a partial list of references 830 to avoid overcrowding the display. The analyst may be provided the option to display all references 830, a specific number of references 830, may be provided the ability to scroll through the list of references 830 using a scroll bar, page through a specific number of references 830 (FIG. 17) or any other conventional method for displaying large numbers of items on a display. As indicated in FIG. 16, the display may also provide the number 840 of remaining references 830 for a particular list of common items of evidence between case files.

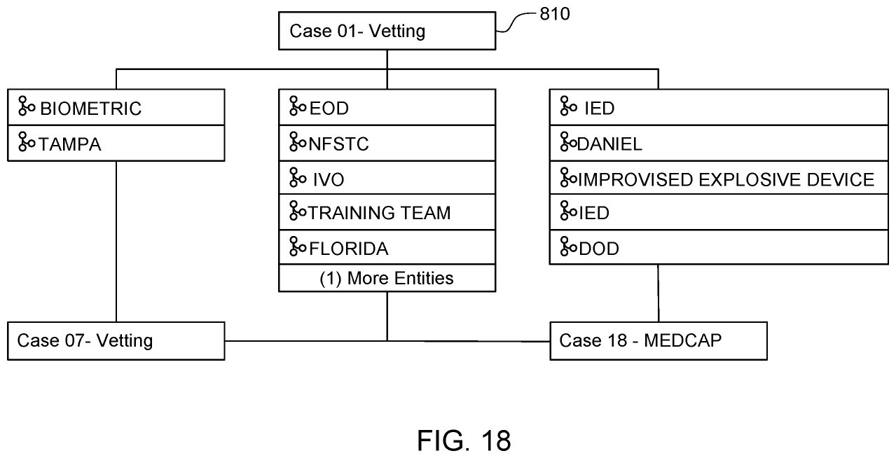

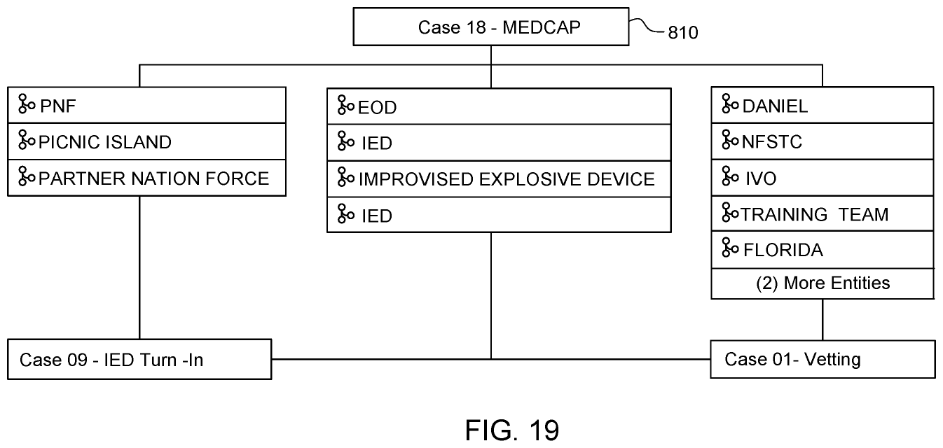

FIG. 18 illustrates that in one or more embodiments, the analyst may further navigate down the chain of potentially related case files, without having to return to the original dashboard 800. As illustrated, by selecting one of the case files (e.g., case 01) the display arranges the selected case (case 01) as the primary or current case 810 and displays other cases that share common patterns or data with case 01 other than the information just reviewed in relation to case 04. Although not illustrated, the display may provide some form of indicator (e.g., a small text box) to the analyst reminding the analyst that the present display originated from a different primary case 810. It may also provide a navigation tool, not illustrated, allowing the analyst to return to the previous display and/or to return to the original dashboard 800. FIG. 19 illustrates that the user may continue to traverse the graph by continuing to select different case files, in this case, case 18.

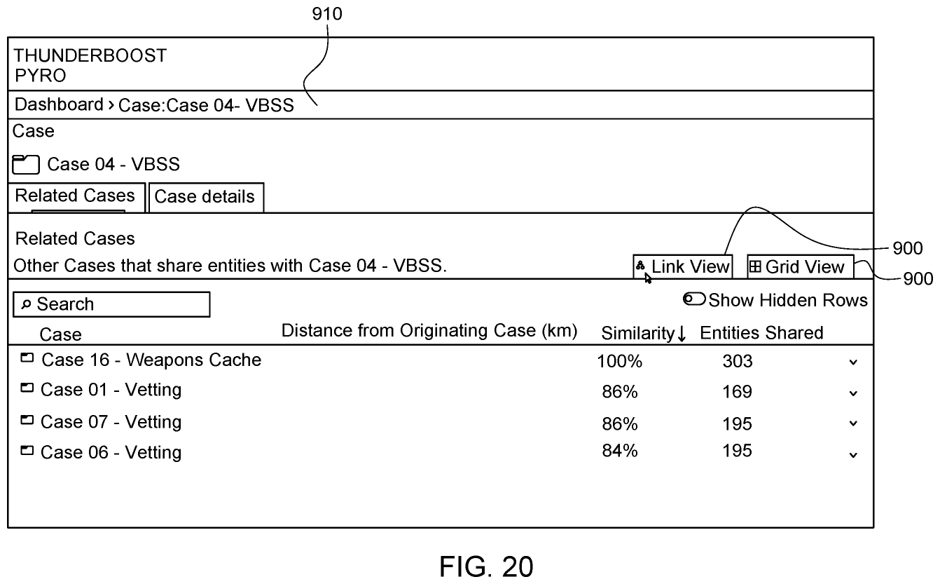

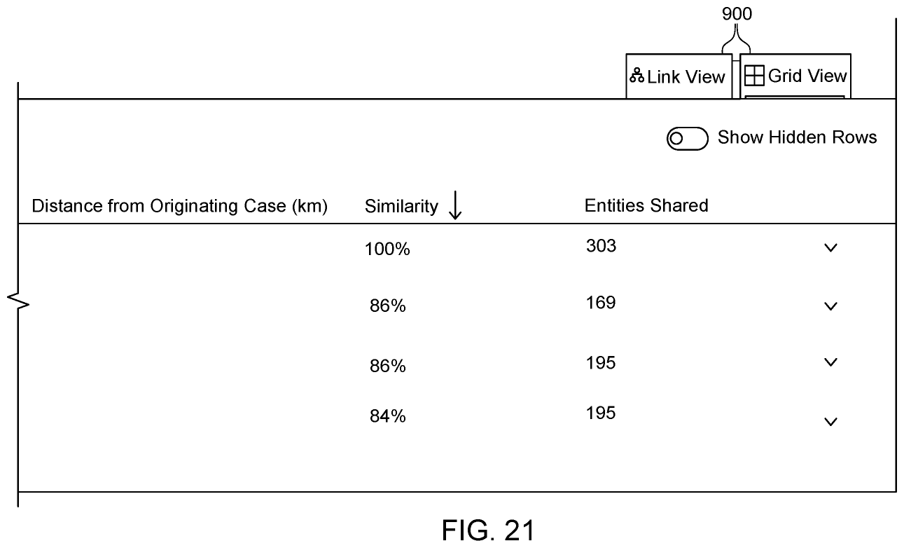

FIGS. 20-24 illustrate that in one or more embodiments, the analyst may be provided the ability to toggle between a link view (FIGS. 14-19) and a grid view. The link view tends to consume more display real estate, which may be problematic on smaller displays. It further places a constraint on how much information can be displayed effectively, such as how many case files can be practically displayed together, whereas the grid view may be more compact. FIG. 20, which provides an exemplary grid view, illustrates tabs 900 which provide the analyst the ability to toggle between views. FIG. 21 merely provides a zoomed-in view of the tabs 900 from FIG. 20.

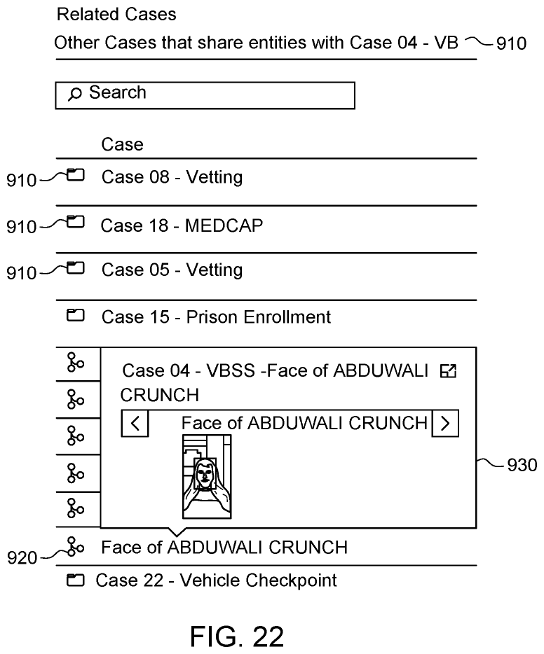

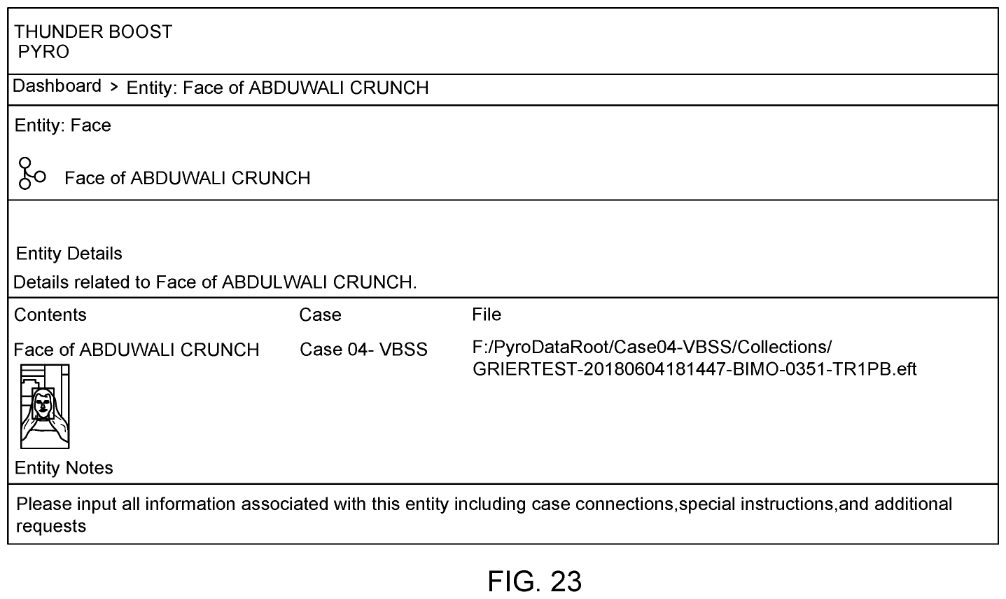

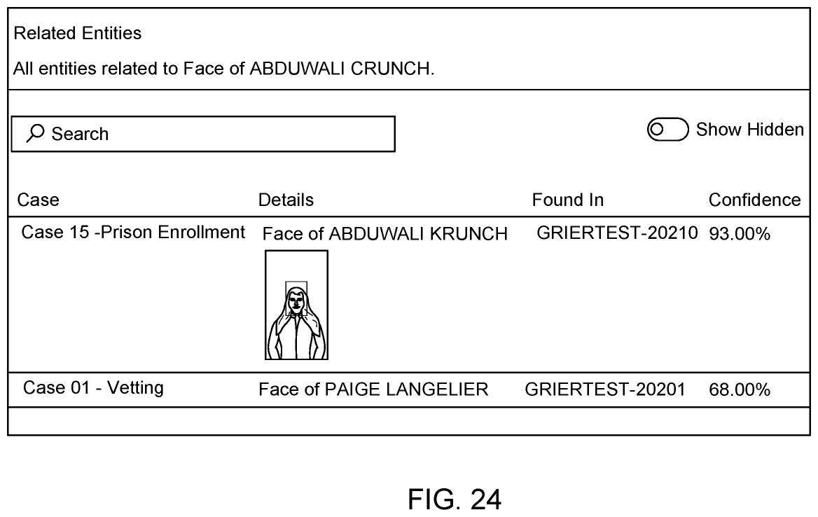

FIGS. 22-24 illustrate and example using the grid view. In this example, the technology performed facial recognition on the data using one or more supervised machine learning plug-in modules. As with the link view, within the grid view (FIG. 22), the analyst may select a particular case file (e.g., case—04) 910, which may trigger the display of other case files 910 that share with case—04 common patterns or data (items of evidence) 920. As illustrated in FIG. 22, a case file may be collapsed to show just the case file number and/or a small number of common items of evidence 920 or expanded to show additional common items of evidence 920. If, as is the case in this example, the analyst selects the item of evidence corresponding to facial recognition evidence 910 (e.g., “Face of ABDUWALI CRUNCH” in case 15) additional information 930 may be displayed (e.g., in a pop-up window, a new window (FIG. 24) or a further expansion of the case info (not shown), etc.) such as the image of Abduwali Crunch. As illustrated in FIG. 23, the analyst may review the information contained in the primary case file to perform a manual comparison as well.

The technology disclosed may leverage a third-party module to extract or recognize a particular face. Similarly, the disclosed technology may use one or more open-source libraries to perform matching between faces that have previously been extracted. Accordingly, in these examples, the disclosed technology may pull or incorporate the identified links or matches between previously recognized faces into the link analysis corresponding to the link view, as further discussed above.

In view of the above, the technology of this application may distinguish from, and improve upon, related technologies that display relationships within a link browser experience, but which do not perform any machine learning-based prioritization or pruning to render the analyst job more efficient and convenient. In other words, the manner of displaying information through the link browser experience or other graphical user interface is rendered more efficient using previously-identified relationships and/or pruning of relevant information through the analysis of previous case files, as further discussed above.

In one or more embodiments, the disclosed technology may leverage unsupervised machine learning, as distinguished from supervised machine learning. A conventional supervised machine learning algorithm may operate upon a curated data set. Thus, a human may be required to analyze the entire data set and tag everything. In various conventional embodiments, the analyst may also be forced to manually clean the data. Additionally, the analyst may be required to perform validation procedures and attempt to ensure that the supervised machine learning protocol is performing accurately. In contrast, the usage of an unsupervised machine learning algorithm may enable the extraction of patterns within data that has not been cleaned and/or has not been tagged. For example, within the context of forensics investigations, such as but not limited to military or law enforcement investigations, the source of data may typically be an adversary, who may be motivated to oppose, or render difficult, the job of the investigator. Accordingly, the corresponding data set may be disorganized and disconnected and not neatly curated. Thus, rather than relying on previously tagged data using a supervised machine learning model, the unsupervised machine learning protocol employed by the disclosed technology may instead attempt to identify relationships or patterns based on the identification of similar relationships (e.g., matching phone numbers to phone numbers, matching faces to faces, matching textual strings to textual strings, etc.).

An advantage of the disclosed technology may be its ability to provide cross-modality functionality. More specifically, conventional technology may attempt, for example, to identify connections between two cell phones. The conventional technology may pull data only from both cell phones, even though various phone numbers may have been previously extracted from other modalities such as documents and biometric enrollments. Accordingly, the conventional technology may attempt to identify matching contacts, call records, between two separate cell phones but will ignore the additional potentially relevant stored information. Thus, conventional technology is limited to analyzing like technologies: i.e., two cell phones. In contrast, in one or more embodiments, the disclosed technology may use heterogeneous data sources, such as but not limited to those used in military intelligence investigations. In these investigations, the technology can match a face from an EBTS (Electronic Biometric Transmission Specification) or biometric enrollment file to a face from another technology (e.g., cellphone photos).

As one illustrative example, a new contractor may need to be vetted, and so his finger printing may be taken, his face may be extracted through facial recognition, and his name may be recorded. In this illustrative example, the technology may match the image of the contractor's face taken through facial recognition to another image of his face that is found on a cell phone that was obtained from an individual who was caught by law enforcement or military personnel attempting to perform a terrorist act, etc. In contrast to the conventional technology (which matches cell phone-extracted data to other cell phone-extracted data), the disclosed technology may match data extracted from one qualitatively distinct entity (e.g., cell phone) to data extracted from another qualitatively distinct entity (e.g., a biometric enrollment file). Similarly, other conventional technology may enable a Marine to extract a fingerprint, at which point the fingerprint may be compared to other similarly collected fingerprints stored within a database. However, this conventional technology does not perform cross-modality analysis.

Having thus described at least one preferred embodiments of the technology, advantages can be appreciated. Variations from the described embodiments exist without departing from the scope of the claims. It is apparent that apparatus, systems and methods are provided that leverage plugin modules supplemented by machine learning to obtains data from one or more data sources, separate the data into smaller units of data (e.g., deconstructs the data into its smallest logical data items/fields) determine common data types within the smaller units, create “edges” that match data from the one or more data sources with data already in the system, then determines potentially overlapping clusters of information to identify potential connections between investigations and display this information in an efficient and navigable manner. Although embodiments have been disclosed herein in detail, this has been done for purposes of illustration only, and is not intended to be limiting with respect to the scope of the claims, which follow. It is contemplated by the inventors that various substitutions, alterations, and modifications may be made without departing from the spirit and scope of the technology as defined by the claims. Other aspects, advantages, and modifications are considered within the scope of the following claims. The claims presented are representative of the technology disclosed herein. Other, unclaimed technology is also contemplated. The inventors reserve the right to pursue such technology in later claims.

Insofar as embodiments of the technology described above are implemented, at least in part, using a computer system, it will be appreciated that a computer program for implementing at least part of the described methods and/or the described systems is envisaged as an aspect of the technology. The computer system may be any suitable apparatus, system or device, electronic, optical, or a combination thereof. For example, the computer system may be a programmable data processing apparatus, a computer, a Digital Signal Processor, an optical computer or a microprocessor. The computer program may be embodied as source code and undergo compilation for implementation on a computer, or may be embodied as object code, for example.

It is also conceivable that some or all functionality ascribed to the computer program or computer system may be implemented in hardware, for example by one or more application specific integrated circuits and/or optical elements. Suitably, the computer program can be stored on a carrier medium in computer usable form, which is also envisaged as an aspect of the technology. For example, the carrier medium may be solid-state memory, optical or magneto-optical memory such as a readable and/or writable disk for example a compact disk (CD) or a digital versatile disk (DVD), or magnetic memory such as disk or tape, and the computer system can utilize the program to configure it for operation. The computer program may also be supplied from a remote source embodied in a carrier medium such as an electronic signal, including a radio frequency carrier wave or an optical carrier wave.

It is accordingly intended that all matter contained in the above description or shown in the accompanying drawings be interpreted as illustrative rather than in a limiting sense. It is also to be understood that the following claims are intended to cover all generic and specific features of the technology as described herein, and all statements of the scope of the technology which, as a matter of language, might be said to fall there between.

Having described the technology, what is claimed as new and secured by Letters Patent is:

1. A computer-implemented method for creating an improved forensic investigation graph, at least a portion of the method being performed by a computing device comprising at least one processor, the method comprising:
clustering nodes of connected data according to a maximal nearest neighbor algorithm to create maximal nearest neighbor clusters; wherein a first node of data is directly connected to at least a second node of data and indirectly connected to a third node of data through the second node; wherein a nearest neighbor includes only sets of nodes that are directly connected; wherein a cluster of data includes combinations of connected nodes; wherein a cluster of nearest neighbors only includes combinations of nodes that are directly connected to each other; and
wherein the maximal nearest neighbor clusters are created by determining all clusters or nearest neighbors and removing all nearest neighbor clusters that are subsets of another nearest neighbor cluster;
and displaying the maximal nearest neighbor clusters on a display associated with the computing device;
wherein the maximal nearest neighbor clusters represent data acquired in the performance of a forensic investigation.

2. The method according to claim 1 further comprising utilizing unsupervised machine learning to generate the maximal nearest neighbor clusters.

3. The method according to claim 1 further comprising utilizing supervised machine learning to generate the maximal nearest neighbor clusters.

4. The method according to claim 1 further including displaying the maximal nearest neighbor clusters in a link view.

5. The method according to claim 1 displaying the maximal nearest neighbor clusters in a grid view.

6. The method according to claim 1 further comprising generating the nodes of connected data by extracting a first data from a first data source data source and extracting a second data from a second data source; deconstructing the first data and the second data into constituent data types and comparing like data types.

7. A non-transitory computer-readable medium comprising one or more computer-executable instructions that, when executed by at least one processor of a computing device, cause the computing device to:
cluster nodes of connected data according to a maximal nearest neighbor algorithm to create maximal nearest neighbor clusters; wherein a first node of data is directly connected to at least a second node of data and indirectly connected to a third node of data through the second node; wherein a nearest neighbor includes only sets of nodes that are directly connected; wherein a cluster of data includes combinations of connected nodes; wherein a cluster of nearest neighbors only includes combinations of nodes that are directly connected to each other; and
wherein the maximal nearest neighbor clusters are created by determining all clusters or nearest neighbors and removing all nearest neighbor clusters that are subsets of another nearest neighbor cluster;
and display the maximal nearest neighbor clusters on a display associated with the computing device.

8. The non-transitory computer-readable medium according to claim 16, wherein the instructions further causing the computing device to employ supervised machine learning to generate the maximal nearest neighbor clusters.

9. The non-transitory computer-readable medium according to claim 16, wherein the instructions further causing the computing device to employ unsupervised machine learning to generate the maximal nearest neighbor clusters.

10. The non-transitory computer-readable medium according to claim 16, the instructions further causing the computing device to display the maximal nearest neighbor clusters in a link view on the display device.

11. The non-transitory computer-readable medium according to claim 16, the instructions further causing the computing device to display the maximal nearest neighbor clusters in a grid view on the display device.
